SEQUENCE LISTING (39 sequences)

SEQ ID NO: 1 - DNA, 22 bp, Artificial Sequence
  Synthetic
atttttttgtttgttggggatt

SEQ ID NO: 2 - DNA, 24 bp, Artificial Sequence
  Synthetic
ggaaatttatttttagtgttgtag

SEQ ID NO: 3 - DNA, 26 bp, Artificial Sequence
  Synthetic
acaaatcatcactctaacaactcaat

SEQ ID NO: 4 - DNA, 22 bp, Artificial Sequence
  Synthetic
aagtttggttatggtggtttta

SEQ ID NO: 5 - DNA, 24 bp, Artificial Sequence
  Synthetic
atcaactaccatcaacttccttac

SEQ ID NO: 6 - DNA, 24 bp, Artificial Sequence
  Synthetic
ttttagtgatattttggygggttg

SEQ ID NO: 7 - DNA, 21 bp, Artificial Sequence
  Synthetic
gggatttgggaaagagggaaa

SEQ ID NO: 8 - DNA, 22 bp, Artificial Sequence
  Synthetic
aacrtcctaaatcccctaaacc

SEQ ID NO: 9 - DNA, 22 bp, Artificial Sequence
  Synthetic
ggttttatgttgggagttttga

SEQ ID NO: 10 - DNA, 19 bp, Artificial Sequence
  Synthetic
tactccctaaaccccrcta

SEQ ID NO: 11 - DNA, 22 bp, Artificial Sequence
  Synthetic
tgggtatgggttagaaggattt

SEQ ID NO: 12 - DNA, 22 bp, Artificial Sequence
  Synthetic
acaccaaaaaaaaaactcatct

SEQ ID NO: 13 - DNA, 26 bp, Artificial Sequence
  Synthetic
attagaaaaagagtttatttgggaaa

SEQ ID NO: 14 - DNA, 22 bp, Artificial Sequence
  Synthetic
ttcctttatccccaatctaaac

SEQ ID NO: 15 - DNA, 18 bp, Artificial Sequence
  Synthetic
tattgcggagtgcgggtc

SEQ ID NO: 16 - DNA, 18 bp, Artificial Sequence
  Synthetic
tcgacgaactcccgacga

SEQ ID NO: 17 - DNA, 25 bp, Artificial Sequence
  Synthetic
aaaacgccctaatccgcatccaacg

SEQ ID NO: 18 - DNA, 24 bp, Artificial Sequence
  Synthetic
tgcgtttatatttagttaatcggc

SEQ ID NO: 19 - DNA, 22 bp, Artificial Sequence
  Synthetic
gaaatacgaatcgaaaaacgaa

SEQ ID NO: 20 - DNA, 20 bp, Artificial Sequence
  Synthetic
acgctccccattcccgtcga

SEQ ID NO: 21 - DNA, 32 bp, Artificial Sequence
  Synthetic
catggtgagcgatgcrddddrgcatcgctcac

SEQ ID NO: 22 - DNA, 100 bp, Artificial Sequence
  Synthetic
ggagagagaagtagttgtgtaatttgttggatgtggattagggtgttttttatttttgttgggagtttgttgattggttgggtgtgggtgtatgtgattg

SEQ ID NO: 23 - DNA, 98 bp, Artificial Sequence
  Synthetic
ggagagagaagtagttgtgtaatttgttggatgtggattagggtgttttttatttttgttgggagtttgttgattggttgggtgtggcgtatgtgatc

SEQ ID NO: 24 - DNA, 103 bp, Artificial Sequence
  Synthetic
ggagagagaagtagttgtgtaattctgttgggatgctggattagggcgttttttattttcgtcgggagttcgtcgattggttgggtgtgggcgtacgtgatcg

SEQ ID NO: 25 - DNA, 101 bp, Artificial Sequence
  Synthetic
ggagagagaagtagttgtgtaattcgttggatgcggattagggcgttttttattttcgtcgggagttcgtcgattggttgggtgtgggcgtacgtgatcga

SEQ ID NO: 26 - DNA, 103 bp, Artificial Sequence
  Synthetic
ggagagagaagtagttgtgtaattcgttggatgcggattagggcgttttttattttcgtcgggagttcgtcgattggttgggtgtgggcgtacgtgatcgata

SEQ ID NO: 27 - DNA, 103 bp, Artificial Sequence
  Synthetic
gtttttgatgggaatggggagtgttttggtttgtatttagtggattatatagttgtttttttttttgttttttgatttgaattttgtagtggggtatagggtt

SEQ ID NO: 28 - DNA, 103 bp, Artificial Sequence
  Synthetic
gttttctgatgggaatggggagcgttttggtttgtatttagtggattatatagttgtttttttttttgttttttgatttgtattttgtagtggggtatagggt

SEQ ID NO: 29 - DNA, 106 bp, Artificial Sequence
  Synthetic
gttttcgacgggaatggggagcgttttggttcgtatttagcggattatatagttgttttttttttcgtttttcgattcgtatttcgtagtggggtatagggtttcg

SEQ ID NO: 30 - DNA, 103 bp, Artificial Sequence
  Synthetic
gttttcgacgggaatggggagcgttttggttcgtatttagcggattatatagttgttttttttttcgtttttcgattcgtatttcgtagtggggtatagggtt

SEQ ID NO: 31 - DNA, 103 bp, Artificial Sequence; identical to SEQ ID NO: 30
  Synthetic

SEQ ID NO: 32 - DNA, 105 bp, Artificial Sequence
  Synthetic
tttttttttttgttttttgatttgtattttgtagtggggtatagggttttgtttaattgtatagtttgtttagttttagttttgtattttttgggggtatgtttt

SEQ ID NO: 33 - DNA, 102 bp, Artificial Sequence
  Synthetic
tttttttttttgttttttgatttgtattttgaaggggggtatagggttttgttaacctgaaaatttggttaattttaatttctgatttttcggggggatgtt

SEQ ID NO: 34 - DNA, 103 bp, Artificial Sequence
  Synthetic
ttttttttttcgtttttcgattcgtatttcgaaggggggtatagggtttcgtttaatcgtatagtttgtttagttttagtttcgtatttttcgggggtatgtt

SEQ ID NO: 35 - DNA, 105 bp, Artificial Sequence
  Synthetic
ttttttttttcgtttttcgattcgtatttcgtagtggggtatagggtttcgtttaatcgtatagtttgtttagttttagtttcgtatttttcgggggtatgtttt

SEQ ID NO: 36 - DNA, 103 bp, Artificial Sequence
  Synthetic
ttttttttttcgtttttcgattcgtatttcgtagtggggtatagggtttcgtttaatcgtatagtttgtttagttttagtttcgtatttttcgggggtatgtt

SEQ ID NO: 37 - DNA, 74 bp, Artificial Sequence
  Synthetic
gtttatatttagttaattggtgggtttttgatgggaatggggagtgttttggtttgtatttagtggattatata

SEQ ID NO: 38 - DNA, 74 bp, Artificial Sequence
  Synthetic
gtttatatttagttaattggtgggtttttgatgggaatggggagtgttttggttcgtatttagcggattatata

SEQ ID NO: 39 - DNA, 74 bp, Artificial Sequence
  Synthetic
gtttatatttagttaatcggcgggttttcgacgggaatggggagcgttttggttcgtatttagcggattatata

(19) United States
(12) Patent Application Publication
(10) Pub. No.: US 2011/0256538 A1
(43) Pub. Date: Oct. 20, 2011

(54) EPIGENOMIC DNA MODIFICATIONS FOR TISSUE TYPING, EARLY CANCER DETECTION, AND DISEASE MANAGEMENT

(75) Inventors: Ying-Hsiu Su, Audubon, PA (US); Surbhi Jain, Lansdale, PA (US)

(73) Assignee: Philadelphia Health & Education Corporation d/b/a Drexel University College of Medicine, Philadelphia, PA (US)

(21) Appl. No.: 13/087,029

(22) Filed: Apr. 14, 2011

Related U.S. Application Data
(60) Provisional application No. 61/324,530, filed on Apr. 15, 2010.

(51) Int. Cl. C12Q 1/68

(52) U.S. Cl. 435/6.11

(57) ABSTRACT

Provided herein is a suitable method for detecting the presence or absence of a cancer in an individual, by determining the level of methylation of the sense strand of a selected regulatory region of a tumor suppressor gene. Also provided herein is a method of detecting the presence or absence a cancer in an individual by determining if there is an apparent 100% methylation by assay of the CpG sites in the anti-sense strand of a selected regulatory region of a tumor suppressor gene. Also provided herein is a method of tissue typing by determining the level of methylation of the anti-sense strand of a selected regulatory region of a tumor suppressor gene indicating an enhanced likelihood that a tissue is liver.

### CROSS-REFERENCE TO RELATED APPLICATIONS

This application claims the benefit of U.S. Provisional Application No. 61/324,530, filed Apr. 15, 2010, the disclosure of which is incorporated herein by reference in its entirety.

### STATEMENT REGARDING FEDERAL RIGHTS

This invention was made with government support under grant numbers NIH RO1 CA125642 and CA146799, awarded by the National Institute of Health and The Early Detection Research Network Grant, awarded by the National Cancer Institute. The government has certain rights in the invention.

### TECHNICAL FIELD

The technical field is tissue typing, early cancer detection, and disease management.

### BACKGROUND

Hepatocellular carcinoma (HCC) is an aggressive widespread malignancy that has a survival rate of 14% (1). Methylation of multiple tumor suppressor genes has been demonstrated to play a role in the pathogenesis of HCC (4-6). Among these multiple tumor suppressor genes, inactivation of the adenomatous polyposis coli (APC) gene by genetic or epigenetic modifications, particularly methylation, is a key causative event in several cancers, including HCC (5, 7-9, 11).

De novo somatically acquired DNA methylation occurs predominantly at CpG dinucleotides within the promoter and at upstream exons of genes and, in conjunction with histone modification, alters chromatin density and the accessibility of DNA to transcriptional cellular machinery, thereby modulating the expression of the underlying DNA sequence (12). DNA methylation patterns are believed to be established early during development and then maintained during somatic divisions (13). Hemimethylated CpG sites occur transiently during developmental processes or during carcinogenesis, wherein either active demethylation or de novo methylation can occur, resulting in gene reactivation or inactivation, respectively (13-18). Sparse strand-independent hemimethylation of human L1 transposable elements (14, 15, 18) and of the fragile X gene FMR1 found in the human genome was thought to be due to infidelity in maintaining a methylated state of cytosine (15).

Although an association between HCC and hypermethylation of the promoter 1A and the first exon of the APC gene (mAPC) has been extensively documented, the degree of this association varies among different studies (5, 19-21) (Table 1). Some studies show up to 81% association of mAPC with HCC and no mAPC in normal livers whereas others report a predominance of mAPC in both HCC and normal liver samples. Most of the studies use methylation-specific PCR (MSP) as the tool for detecting methylation. The primary variable in these studies is the location of the MSP primers; some target the sense strand, and others target the antisense strand (Table 1).

TABLE 1
Comparison of primer locations and association between APC promoter
methylation and HCC in previously published literature.

| | | | Methylation in | |
|---|---|---|---|---|
| | | | Noncancerous | Methylation |
| | Primer | | Liver Tissue | in HCC |
| Study | Location | Technique | % (n) | % (n) |
| 1(4) | Sense | MSP | 0 (20) | 81.7 (60) |
| 2(23) | Sense | MSP | 14.28 (14) | 53 (51) |
| 3(6) | Sense | MSP | NA (95% specificity, n = 20 for | |
| | | | HCC and non-HCC tissue) | |
| 4(21) | Antisense | Methylight- | 81 (19) | 100 (19) |
| | | Taqman | | |
| 5(20) | Antisense | Quantitative | 100 (16) | 100 (34) |
| | | MSP | | |

HCC, hepatocellular carcinoma; MSP, methylation-specific polymerase chain reaction; NA, not applicable.

Thus, there remains a need for a method to more consistently and accurately determine the hypermethylation state of the APC1 promoter for tissue typing, early disease detection, and disease management.

### SUMMARY

Provided herein is a suitable method for detecting the presence or absence of a cancer in an individual by determining the level of methylation of the sense strand of a selected regulatory region of a tumor suppressor gene from the individual and comparing the level of methylation with the level of methylation found in one or more control samples from individuals known not to have the cancer and correlating a finding of elevated methylation in the individual as compared to the level of methylation in one or more controls with an enhanced likelihood that the individual has cancer. Also provided herein is a suitable method for detecting the presence or absence of a cancer in an individual by determining if there is an apparent 100% methylation by assay of CpG sites in the anti-sense strand of a selected regulatory region of a tumor suppressor gene from the individual and correlating a finding of an apparent 100% methylation by assay in the individual with an enhanced likelihood that the individual has cancer. Also provided herein is a suitable method for tissue typing by determining the level of methylation of the anti-sense strand of a selected regulatory region of a tumor suppressor gene and comparing the level of methylation with the level of methylation found in one or more control samples and correlating a finding of elevated methylation as compared to the level of methylation in the one or more controls with an enhanced likelihood that a tissue is liver.

### BRIEF DESCRIPTION OF THE DRAWINGS

FIG. 1(A) shows a diagram of the locations of bisulfite sequencing primers and the CpG sites, indicated by the vertical bars, in the promoter and the first exon regions of the APC gene (Genebank accession # NG_{—}0084811, nt. 34,793-36,093). The ATG site is also indicated. The CpG sites bracketed by the bisulfite sequencing primers for the sense strand (APC-S) and the antisense strand (APC-AS) were numbered from 1 to 30 on the basis of the sense strand 5′ to 3′ direction. FIG. 1(B-D) shows chromatograms showing the sequencing data for the 3 pairs of bisulfite specific primers (B) APC-S, (C) APC-AS-1, (D) APC_AS-2. Rows show the sequencing data from spiked standards. Row 1 has 100% unmethylated DNA. Row 2 has 10% methylated DNA in a background of unmethylated DNA. Row 3 has 25% methylated DNA in a background of unmethylated DNA. Row 4 has 50% methylated DNA in a background of unmethylated DNA. Row 5 has 100% methylated DNA. FIG. 1(E) shows the index for analysis of the methylation status of each CpG site based on the DNA sequencing chromatograms obtained from heart, liver, and one liver bisulphite-specific PCR clone, ranging from CpG site #8 to #14. At each CpG site, four results are possible: (1) only C was detected (C only, black box, indicating methylation); (2) the C peak was higher than the T peak (C>T, hatched box); (3) the C peak was equal to or lower than the T peak (C=T & C<T, dotted box); and (4) only T was detected (T only, open box, indicating no methylation). FIG. 1(F) shows the index used to analyze the sequencing data based on FIG. 1 (B-E).

FIG. 2 shows the methylation status of each CpG site in both sense (S) and antisense (AS) strands of the promoter and the first exon region of the APC gene in hepatocellular carcinoma (HCC) tissue, matched adjacent non-HCC liver tissue (Adj. Non-HCC) (FIG. 2(A)), and normal (FIG. 2(B)), hepatitis-infected (hepatitis) (FIG. 2(C)), and cirrhotic liver (cirrhosis) (FIG. 2(D)) tissues. The data were analyzed as described in FIGS. 1(E) and (F). Because of the large amount of T in the DNA template after bisulfite conversion, sequencing results from some CpG sites were not available and are designated as x.

FIG. 3(A) shows serial 1:10 dilutions of human methylated bisulfite converted genomic DNA were amplified as per the description of APC sense MSP assay. The figure shows amplification curves above and the standard curve is shown below. FIG. 3(B) shows spiked methylated DNA standards in background of unmethylated DNA were amplified as per the description of APC sense MSP assay. FIG. 3(B) shows amplification curves above and the standard curve is shown below. FIG. 3(C) shows serial 1:10 dilutions of human methylated bisulfite converted genomic DNA were amplified as per the description of APC anti-sense MSP assay. FIG. 3(C) shows amplification curves above and the standard curve is shown below. FIG. 3(D) shows spiked methylated DNA standards in background of unmethylated DNA were amplified as per the description of APC anti-sense MSP assay. FIG. 3(D) shows amplification curves above and the standard curve is shown below.

FIG. 4 shows a graph showing the comparison between percentage of methylation positive samples obtained from different liver tissue samples as labeled analyzed by APC sense and anti-sense strand MSP.

FIG. 5 (A) shows the methylation density of the antisense strand of the APC promoter and first exon region of HCC compared with normal liver tissue and HCC compared with adjacent matched non-HCC by BSP direct sequencing and BSP cloning and sequencing. (A) Histogram comparing the density of mCpG detected in the antisense (AS) strand of the APC gene in HCC vs. adjacent non-HCC (p<0.0001), and HCC vs. normal liver (***p<0.0001) tissue by BSP direct sequencing analyzed as described in FIG. 1(B). (FIG. 5(B)) Comparison of the overall methylation density (percent of methylation) of each CpG site of HCC compared with adjacent non-HCC (***p<0.0001) tissue, and HCC compared with normal liver (p<0.0001) tissue by BSP cloning and sequencing as detailed in Example 1. The methylation density for each CpG site of each DNA sample is summarized in FIG. 6.

FIG. 6 shows methylation density of each CpG site obtained by DNA sequencing of each BSP clone. An example of DNA sequencing results obtained from 14 BSP clones isolated from cloning the BSP product derived from normal liver sample-2 (A), HCC-sample 6 (B), and adjacent non-HCC sample 6 (C). The percent of methylation for each CpG site was calculated using the number of mCpGs detected per total number of clones analyzed, as listed at the bottom of the figure. (D) Summary of the percent of methylation for each CpG site from DNA isolated from 4 normal livers, 4 HCC samples, and the matched adjacent non-HCC tissue. (E) Histogram showing the percentage of each CpG site methylation for all CpG sites as tabulated in (D) (***P<0.0001 for HCC vs normal liver and ***P<0.0001 for HCC vs adjacent non-HCC).

FIG. 7 shows a schematic showing the 3 hemimethylated molecules obtained by cloning and sequencing of hairpin bisulfite PCR product of one adjacent non-HCC tissue. The numbers on left indicate the number of methylated CpG sites out of total 23 sites analyzed. The top strand is sense strand (S) and the bottom is anti-sense strand (AS) linked by the Hairpin linker molecule.

FIG. 8 shows methylation profiles for sense (S) and antisense (AS) strands of the promoter and the first exon regions of the APC gene in nonliver normal tissues. DNA sequencing data were obtained by BSP sequencing and analyzed as described in FIGS. 1(E) and (F).

FIG. 9 shows the methylation profiles of the sense (S) and antisense (AS) strands of the promoter and the first exon regions of the GSTP-1 gene (A), the beta actin gene (B), and the APC gene (E) by BSP direct sequencing and analyzed as described in FIGS. 1(E) and (F) for DNA isolated from normal liver tissue (A,B); HCC and adjacent non-HCC tissue (Adj) (D); other normal non liver tissues (E); and liver tissue from three mice (C).

### DETAILED DESCRIPTION OF ILLUSTRATIVE EMBODIMENTS

The present subject matter may be understood more readily by reference to the following detailed description taken in connection with the accompanying examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it is understood that the particular value forms another embodiment. All ranges are inclusive and combinable.

Examples are provided to assist in a further understanding of the inventions. Particular materials used, protocols and conditions are intended to be further illustrative of the inventions and should not be construed to limit the reasonable scope thereof.

Provided herein is a suitable method for detecting the presence or absence of a cancer in an individual by determining the level of methylation of the sense strand of a selected regulatory region of a tumor suppressor gene from the individual and comparing the level of methylation with the level of methylation found in one or more control samples from individuals known not to have the cancer and correlating a finding of elevated methylation in the individual as compared to the level of methylation in one or more controls with an enhanced likelihood that the individual has cancer. The cancer can be hepatocellular carcinoma (HCC) and the control can be matched adjacent non-HCC sample. The tumor suppressor gene can be adenomatous polypsis coli (APC). The regulatory region can be the promoter of the APC gene, the first exon of the APC gene, or both. The individual can be a human.

The level of methylation of the sense strand can be determined by methylation specific PCR (MSP) and the MSP can use primers of the nucleotide sequence as set forth in SEQ ID NO: 15 and SEQ ID NO:16 and can be probed by a probe of the nucleotide sequence as set forth in of SEQ ID NO:17. The level of methylation of the sense strand can also be determined by bisulfite specific PCR (BSP) and sequencing. The BSP can use primers of the nucleotide sequence as set forth in SEQ ID NO: 1 and SEQ ID NO:2.

Also provided herein is a suitable method for detecting the presence or absence of a cancer in an individual by determining if there is an apparent 100% methylation by assay of CpG sites by assay in the anti-sense strand of a selected regulatory region of a tumor suppressor gene from the individual and correlating a finding of an apparent 100% methylation by assay in the individual with an enhanced likelihood that the individual has cancer. The cancer can be hepatocellular carcinoma (HCC). The tumor suppressor gene can be adenomatous polypsis coli (APC). The regulatory region of the APC gene can be the promoter and first exon of APC. The individual can be a human.

The level of methylation of the anti-sense strand can be determined by bisulfite specific PCR (BSP) and sequencing and the BSP can use primers of the nucleotide sequence as set forth in SEQ ID NO: 3 and SEQ ID NO: 4. Alternatively, BSP can use primers of the nucleotide sequence as set forth in SEQ ID NO: 5 and SEQ ID NO: 6.

Also provided herein is a suitable method for tissue typing by determining the level of methylation of the anti-sense strand of a selected regulatory region of a tumor suppressor gene and comparing the level of methylation with the level of methylation found in one or more control samples and correlating a finding of elevated methylation as compared to the level of methylation in the one or more controls with an enhanced likelihood that a tissue is liver. The tumor suppressor gene can be adenomatous polypsis coli (APC).

Methylation of the promoter of the tumor suppressor, the adenomatous polyposis coli (APC) gene, has been associated with various malignancies, including hepatocellular carcinoma (HCC). However, its specificity to HCC compared to normal liver tissue is unclear. Previously, it has generally been assumed that methylation occurs symmetrically in both strands of DNA. During analysis of the methylation status of both sense and antisense DNA strands from the promoter and the first exon region of the APC gene in samples from HCC tissue, matched adjacent non-HCC tissue, hepatitis-infected, cirrhotic, and normal liver tissue, as well as fetal liver and 12 nonhepatic normal tissues, a novel liver-specific, antisense strand-restricted CpG hemimethylation was discovered. Methylation of only the sense strand of the APC promoter region occurred exclusively in HCC. The density of antisense strand methylation of APC was significantly elevated in HCC compared to other liver tissues (P<0.0001). This strand-specific CpG hemimethylation was not found in the glutathione S-transferase P-1 gene, another HCC-associated tumor suppressor gene, nor was it found in beta-actin in the liver. Moreover, strand-specific hemimethylation of the APC gene did not occur in murine liver tissues. This finding represents the first antisense-specific hemimethylation of a CpG island to be described within the human genome and it appears to be liver-specific.

HCC is a heterogenous tumor with numerous genetic and epigenetic drivers that are not yet completely understood (2, 3). Antisense strand biased methylation in the APC promoter region in liver and demonstrate evidence that, anti-sense strand is found to be frequently methylated in hepatitis, cirrhosis, adjacent non-HCC and HCC tissues whereas the sense strand gets methylated only in HCC. The density of antisense strand methylation of APC was significantly elevated in HCC relative to other liver tissues (P<0.0001). The methylation status of the promoter and the first exon of the APC by bisulphite-specific PCR (BSP) direct sequencing for both sense and antisense DNA strands was analyzed comparing HCC tissue with matched adjacent non-HCC (n=32) liver tissues, patients with cirrhosis (n=5) and hepatitis (n=4) as disease progression controls and from individuals with normal livers (n=6). The results indicated a novel liver-specific epigenetic pattern: an antisense strand-biased CpG hemimethylation of the promoter and first exon of the APC gene. This strand biased methylation phenomenon by methylation specific PCR was confirmed. This strand-specific CpG hemimethylation was not found in the glutathione S-transferase P (GSTP)-1 gene, another HCC-associated tumor suppressor gene, nor was it found in beta-actin in the liver. Methylation of the sense strand of APC occurred exclusively in HCC.

### EXAMPLES

### Example 1

### Study Subjects and Methods

### Subjects

The 32 HBV-related HCC tissues and the adjacent non-HCC liver samples used in this study for BS-PCR DNA sequencing were obtained with informed consent from patients who underwent radical resection at Zhong Shan Hospital, Shanghai, China, and from The National Cheng-Kung University Medical center in accordance with the guidelines of the institutional review board. DNA from normal liver (N2-N6), hepatitis-infected (H1-4), cirrhotic (C1-C6), esophageal, and colon tissues was obtained from The Johns Hopkins University School of Medicine in accordance with The Johns Hopkins University Institutional Review Board protocols. Normal liver N1, heart, and lung tissue samples obtained from the National Disease Research Interchange, Philadelphia were given to us by Immunotope, Inc (Doylestown, Pa.), and normal peripheral blood mononuclear cell DNA was obtained as a gift from the laboratory of Dr. Pooja Jain (Drexel University College of Medicine). Normal liver N2 tissue DNA was purchased from Capital Biosciences (Rockville, Md.), and stomach 1-4, pancreas, kidney, spleen, breast, brain, trigeminal ganglion, and fetal liver DNA was purchased from Biochain (Hayward, Calif.). The individual subject information is listed in Table 2. The mouse livers of Balb/c female mice were obtained from Charles River Laboratories (Wilmington, Mass.).

TABLE 2
Subject information.

| | | Age, | | HBV- | HCV- |
|---|---|---|---|---|---|
| Subject | Gender | years | Pathological Profile | infected | infected |
| N1 | M | 57 | Normal liver | − | − |
| N2 | M | 59 | Normal liver | − | − |
| N3 | M | 66 | Normal liver with concomitant | − | − |
| | | | cholangiocarcinoma | | |
| N4 | F | 78 | Normal liver with concomitant | − | − |
| | | | cholangiocarcinoma | | |
| N5 | F | 62 | Normal liver with concomitant | − | − |
| | | | cholangiocarcinoma | | |
| N6 | M | 61 | Normal liver with concomitant | − | − |
| | | | cholangiocarcinoma | | |
| H1 | F | 50 | Hepatitis, noncirrhotic, adjacent to | − | − |
| | | | multifocal HCC | | |
| H2 | M | 73 | Hepatitis, noncirrhotic, adjacent to HCC | − | − |
| H3 | F | 47 | Hepatitis, noncirrhotic liver adjacent to | − | − |
| | | | HCC | | |
| H4 | M | 70 | Hepatitis, noncirrhotic, adjacent to | − | − |
| | | | recurrent HCC | | |
| C1 | M | 58 | HCV-cirrhosis, adjacent to HCC | − | + |
| C2 | F | 15 | HCV-cirrhosis adjacent to HCC | − | + |
| C3 | M | 60 | Cryptogenic cirrhosis, adjacent to HCC | − | − |
| C4 | F | 67 | HCV-cirrhosis, adjacent to HCC | − | + |
| C5 | M | 57 | HCV-cirrhosis, adjacent to HCC | − | + |
| C6 | M | 51 | HCV-cirrhosis adjacent to multifocal HCC | − | + |
| HCC 1 | F | 59 | HCC | + | − |
| HCC 2 | M | 44 | HCC | + | − |
| HCC 3 | F | 69 | HCC | − | + |
| HCC 4 | M | 69 | HCC | + | + |
| HCC 5 | F | 57 | HCC | − | + |
| HCC 6 | F | 76 | HCC | − | + |
| HCC 7 | M | 54 | HCC | − | − |
| HCC 8 | M | 44 | HCC | + | − |
| HCC 9 | M | 74 | HCC | NA | + |
| HCC 10 | M | 73 | HCC | − | + |
| HCC 11 | F | 30 | HCC | + | NA |
| HCC 12 | M | 48 | HCC | + | NA |
| HCC 13 | F | 55 | HCC | + | NA |
| HCC 14 | M | 50 | HCC | + | NA |
| HCC 15 | M | 68 | HCC | + | NA |
| HCC 16 | M | 62 | HCC | + | NA |
| HCC 17 | F | 57 | HCC | + | − |
| HCC 18 | M | 62 | HCC | + | − |
| HCC 19 | M | 71 | HCC | NA | − |
| HCC 20 | M | 50 | HCC | + | − |
| HCC 21 | F | 51 | HCC | − | − |
| HCC 22 | M | 65 | HCC | + | − |
| HCC 23 | M | NA | HCC | + | NA |
| HCC 24 | M | 61 | HCC | + | − |
| HCC 25 | F | 75 | HCC | + | − |
| HCC 26 | F | 62 | HCC | NA | + |
| HCC 27 | M | 51 | HCC | + | NA |
| HCC 28 | F | 59 | HCC | + | + |
| HCC 29 | M | 56 | HCC | + | NA |
| HCC 30 | F | 42 | HCC | − | − |
| HCC 31 | M | 44 | HCC | − | − |
| HCC 32 | M | 61 | HCC | + | − |
| Pancreas | M | 27 | Normal | NA | NA |
| Kidney | F | 63 | Normal | NA | NA |
| Spleen | F | 83 | Normal | NA | NA |
| Stomach 1 | M | 27 | Normal | NA | NA |
| Stomach 2 | M | 50 | Normal | NA | NA |
| Stomach 3 | M | 58 | Normal | NA | NA |
| Stomach 4 | M | 29 | Normal | NA | NA |
| Brain 1 | M | 78 | Normal | NA | NA |
| Brain 2 | M | 75 | Normal | NA | NA |
| Lung | F | 50 | Normal | NA | NA |
| Heart | M | 67 | Normal | NA | NA |
| Colon 1 | F | 85 | Normal | NA | NA |
| Colon 2 | F | 77 | Normal | NA | NA |
| Esophagus | M | 73 | Normal | NA | NA |
| Trigeminal | M | 75 | Normal | NA | NA |
| ganglion | | | | | |
| Breast 1 | F | 78 | Normal | NA | NA |
| Breast 2 | F | 21 | Normal | NA | NA |
| Fetal Liver | M | 29 | Normal | NA | NA |
| | | weeks | | | |

F, female; HCC, hepatocellular carcinoma; M, male; NA, not applicable.

Table 3 is a summary of all the study subjects including the HCC used for the MSP assay.

TABLE 3
Summary of all subjects information.

| | Normal | Hepatitis | Cirrhosis | HCC |
|---|---|---|---|---|
| Total | 6 | 39 | 41 | 139 |
| Age | 64 ± 7.5 | 55 ± 11.9 | 56 ± 14.4 | 60 ± 11.6 |
| Males | 4 | 19 | 27 | 92 |
| Females | 2 | 20 | 14 | 47 |
| HBV+ | 0 | 12 | 6 | 74 |
| HCV+ | 0 | 31 | 22 | 36 |

### DNA Isolation and Bisulphite Treatment

DNA was isolated by using the Qiagen DNAeasy Blood and Tissue Kit™ (Qiagen, Valencia, Calif.) according to the manufacturer's instructions. The DNA concentration was measured using a Nanodrop 1000™ spectrophotometer (Thermo Fisher Scientific Inc, Wilmington, Del.) at 260 nm absorbance. Bisulfite treatment was performed using Qiagen Epitect Bisulfite conversion Kits™ (Qiagen) following the guidelines of the manufacturer.

### BSP and BSP Direct Sequencing

Bisulfite specific primers were designed using Methyl Express Software™ (ABI, Foster City, Calif.) to amplify the promoter region of the APC, GSTP1, and actin genes for both the sense and antisense strands; the primer sequences are described in Table 4.

Table 4. Primer and probe sequences used for bisulfite DNA sequencing and methylation specific PCR for both sense and antisense DNA strands (Genbank accession number: APC: NG_{—}0084811; GSTP-1: M24485; Actin: NT_{—}007819)

| Assay | Primers and probe sequence | | Strand | Location |
|---|---|---|---|---|
| APC_BSP_S | F: | atttttttgtttgttggggatt | S | 35105-35585 |
| | | (SEQ ID NO: 1) | | |
| | R: | ggaaatttatttttagtgttgtag | | |
| | | (SEQ ID NO: 2) | | |
| APC_BSP_AS1 | F1: | acaaatcatcactctaacaactcaat | AS | 34809-35346 |
| | | (SEQ ID NO: 3) | | |
| | R1: | aagtttggttatggtggtttta | | |
| | | (SEQ ID NO. 4) | | |
| APC_BSP_AS2 | F2: | atcaactaccatcaacttccttac | AS | 35101-35383 |
| | | (SEQ ID NO: 5) | | |
| | R2: | ttttagtgatattttggygggttg | | |
| | | (SEQ ID NO: 6) | | |
| GSTP1_BSP_S | F: | gggatttgggaaagagggaaa | S | 999-1387 |
| | | (SEQ ID NO: 7) | | |
| | R: | aacrtcctaaatcccctaaacc | | |
| | | (SEQ ID NO: 8) | | |
| GSTP1_BSP_AS | F: | ggttttatgttgggagttttga | AS | 980-1312 |
| | | (SEQ ID NO: 9) | | |
| | R: | tactccctaaaccccrcta | | |
| | | (SEQ ID NO: 10) | | |
| Actin_BSP_S | F: | tgggtatgggttagaaggattt | S | 5557986-5558399 |
| | | (SEQ ID NO: 11) | | |
| | R: | acaccaaaaaaaaaactcatct | | |
| | | (SEQ ID NO: 12) | | |
| Actin_BSP-AS | F: | attagaaaaagagtttatttgggaaa | AS | 5557592-5558936 |
| | | (SEQ ID NO: 13) | | |
| | R: | ttcctttatccccaatctaaac | | |
| | | (SEQ ID NO: 14) | | |
| APC_MSP_S | F: | tattgcggagtgcgggtc | S | 35,205-35,222 |
| | | (SEQ ID NO: 15) | | |
| | R: | tcgacgaactcccgacga | | |
| | | (SEQ ID NO: 16) | | |
| | Probe: | 5′-FAM-aaaacgccctaatccgcatccaacg-BHQ1-3′ | | |
| | | (SEQ ID NO: 17) | | |
| APC_MSP_AS | F: | tgcgtttatatttagttaatcggc | AS | 35,246-35,321 |
| | | (SEQ ID NO: 18) | | |
| | R: | gaaatacgaatcgaaaaacgaa | | |
| | | (SEQ ID NO: 19) | | |
| | Probe: | 5′-FAM-acgctccccattcccgtcga-BHQ1-3′ | | |
| | | (SEQ ID NO: 20) | | |

APC, adenomatous polyposis coli; AS, antisense; GSTP-1, glutathione S-transferase
P-1; S, sense

PCR was performed in an Eppendorf Mastercycler Thermocycler™ for 40 cycles with hot-start Taq Polymerase™ (Qiagen). The PCR program started with activation of the polymerase at 95° C. for 15 minutes followed by denaturation at 95° C. for 30 seconds, annealing at the respective annealing temperature for 30 seconds, and extension at 72° C. for 30 seconds, followed by a final 4-minute extension at 72° C. and cooling at 4° C. for all primer sets. The reaction was assembled in a final volume of 20 μl containing 0.5 U HotStart Taq™ (Qiagen), 1×PCR buffer, 200 μM of dNTPs, 0.5 μM of each primer, and bisulphite-treated DNA templates. PCR products were run on 1% agarose gel with 1×TAE buffer. The PCR product of the correct size was excised, and the gel was purified with Qiagen Gel Purification Kit™ (Qiagen) and sent with the appropriate primer for sequencing to the NAPcore facility at the Children's Hospital of Philadelphia, Philadelphia, Pa. Sequencing results were analyzed using ClustalW Software™ (available at http://www.ch.embnet.org/), the Chromas 2.3 Software™ (Technelysium, Tewantin, Queensland, Australia) and Finch TV version 1.4.0™ (Geospiza Inc, Seattle, Wash.).

### BSP Cloning and Sequencing

BSP cloning and sequencing were done for four normal liver (N2, N3, N5 and N6) samples and four HCC and matched adjacent non-HCC bisulphite-treated DNA samples (HCC 2-4, HCC6, non-HCC 2-4, non-HCC 6). The BSP product obtained from APC_F1R1/F2R2 was gel purified using a Qiagen Gel Purification Kit™ (Qiagen) followed by a polishing, ligation, and transformation performed according to the protocols of the PCR-script Amp Cloning Kit™ (Stratagene, Santa Clara, Calif.). The white colonies were screened for the insert using T3, T7 primers. The PCR product obtained from each positive clone thus isolated was then gel purified using a Qiagen Gel Purification Kit™ (Qiagen) and sent for sequencing to the NAPcore facility at the Children's Hospital of Philadelphia. Sequencing results were analyzed using ClustalW Software™ (available at http://www.ch.embnet.org/) and the Chromas 2.3 Software™ (Technelysium). Approximately 7 to 19 clones were sequenced for each sample.

### Methylation Specific PCR

Two quantitative real time PCR assays were developed with primer pairs and Taqman probes as shown in Table 4. For the APC-sense-MSP, A 10-μl reaction was assembled using the FastStart TaqMan Probe Master™ (Roche Applied Science, Mannheim, Germany). The reaction contained 1× FastStart TaqMan Probe Master™, 1.0 μM primers, 2.5 mM MgCl₂, and the DNA template. Using the Roche Light Cycler 480 Real-Time PCR System™, the PCR reaction was performed under the following conditions: 95° C. 10 min, (95° C. 10 s, 65° C. 30 s, 72° C. 10 s)×50 cycles, 40° C. 30 s. For the APC-antisense-MSP, A 10-μl reaction was assembled using the Light Cycler Taqman Master™ (Roche Applied Science, Mannheim, Germany). The reaction contained 1× Taqman Master Mix™, 1.0 μM primers, 2.5 mM MgCl₂, and the DNA template. Using the Roche Light Cycler 2.0 Real-Time PCR System™, the PCR reaction was performed under the following conditions: 95° C. 10 min, (95° C. 10 s, 56° C. 15 s, 72° C. 10 s)×50 cycles, 40° C. 30 s.

### Hairpin-Bisulfite PCR

The hairpin bisulphite PCR was carried out using the guidelines in Laird et al and Hansen et al (15, 16). 2 μg of genomic DNA of interest was first digested with 15 units each of NcoI and PstI enzyme NEB (Ipswich, Mass.) in a 30 μl reaction with 1×NEB buffer 3, 0.1 μg/μl BSA and water at 37° C. for 4 hours. The hairpin-linker (5′-catggtgagcgatgcRDDDDRgcatcgctcac-3) (SEQ ID NO: 21) with staggered ends complementary to the NcoI targeted cut-site of the DNA, was suspended in linker buffer (50 mM Tris, 1 mM EDTA, 100 mM NaCl) to make a 50 uM solution and annealed by running the reaction in a thermocycler at 95° C. 2 mins, 52° C. 10 mins, 4° C. hold. 4 μl of this annealed linker was then phosphorylated in a 20 μl reaction with 10 units of polynucleotide kinase enzyme, 1×NEB PNK Buffer A, and 1 mM RATP and was run at 37° C. 30 mins, 65° C. 15 mins and 4° C. hold. The next step was an overnight ligation of the digested genomic DNA and phosphorylated linker (1:100 ratio) with 20 units/μl T4 DNA ligase, 1×T4 DNA ligase buffer in a 20 μl reaction. This 20 μl ligated product was now treated with the Epitect bisulphite conversion kit and eluted in 40 μl DNA. This bisulphite treated DNA was then used as template for PCR amplification with designed primers APC BSP S (forward) SEQ ID NO:1 and APC BSP AS (reverse) SEQ ID NO 6. The PCR was run at 95° C. 5 mins, (95° C. 1 min, 55° C. 1 min, 72° C. 1 min) for 40 cycles, 72° C. 8 mins, and 4° C. The PCR products were then subsequently cloned using the TOPO TA™ cloning kit K4500-01 (Invitrogen). The well isolated colonies were subjected to colony PCR using insert specific primers to identify positive clones. If the colony PCR product had one specific band at the right size, this PCR product was subsequently sequenced. If the colony PCR showed non-specific bands in addition to right sized bands, minipreps were performed and plasmid DNA was further subjected to PCR amplification and then sequenced.

### Statistical Analysis

The methylation density analysis for BSP direct sequencing and BSP cloning and sequencing was statistically evaluated using a two-sided Pearson χ² test to compare HCC with adjacent normal liver and HCC with normal liver. Contingency tables were constructed for each comparison group (e.g., HCC compared with adjacent normal liver) containing the total number of sites in each of the four methylation density groups (C only, C>T, C<T, T only). For BSP direct sequencing, analysis was done in two ways: (i) including the data for all available CpG sites and (ii) ignoring CpG sites that had data unavailable for any of the samples. For HCC compared with normal liver, sites 3 to 6 and 10 to 17 were used; for HCC compared with adjacent non-HCC, sites 2 to 17 were used for this analysis. For BSP cloning and sequencing methylation density analysis, the total number of methylated CpG sites for each tissue group (HCC vs. normal liver and HCC vs. adjacent non-HCC) were compared using the Pearson χ² test.

### Example 2

### Antisense Strand-Biased Hemimethylation of the Promoter and First Exon Region of the APC Gene in Human Liver Tissue by Bisulfite Specific PCR Sequencing

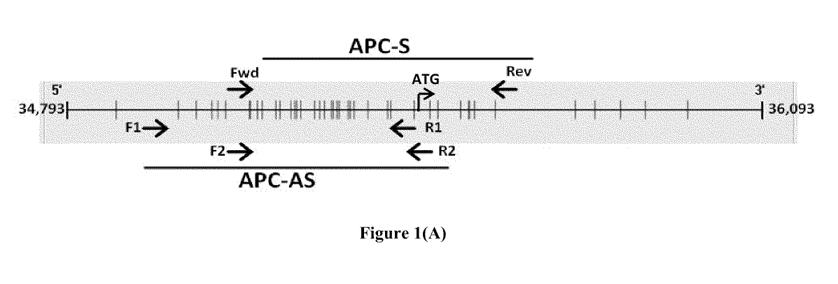

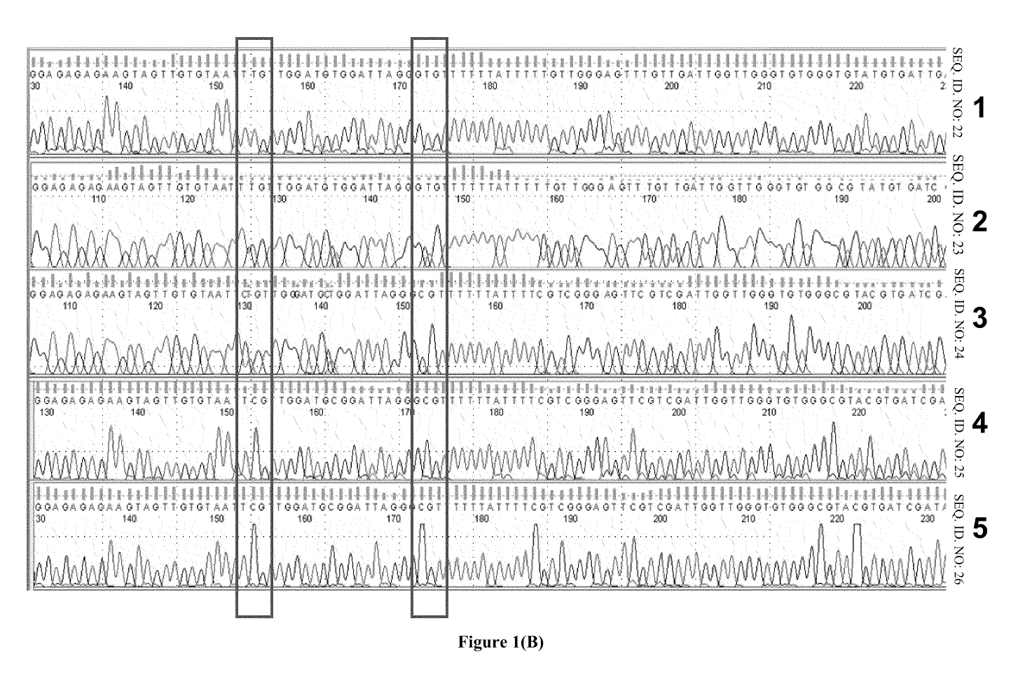

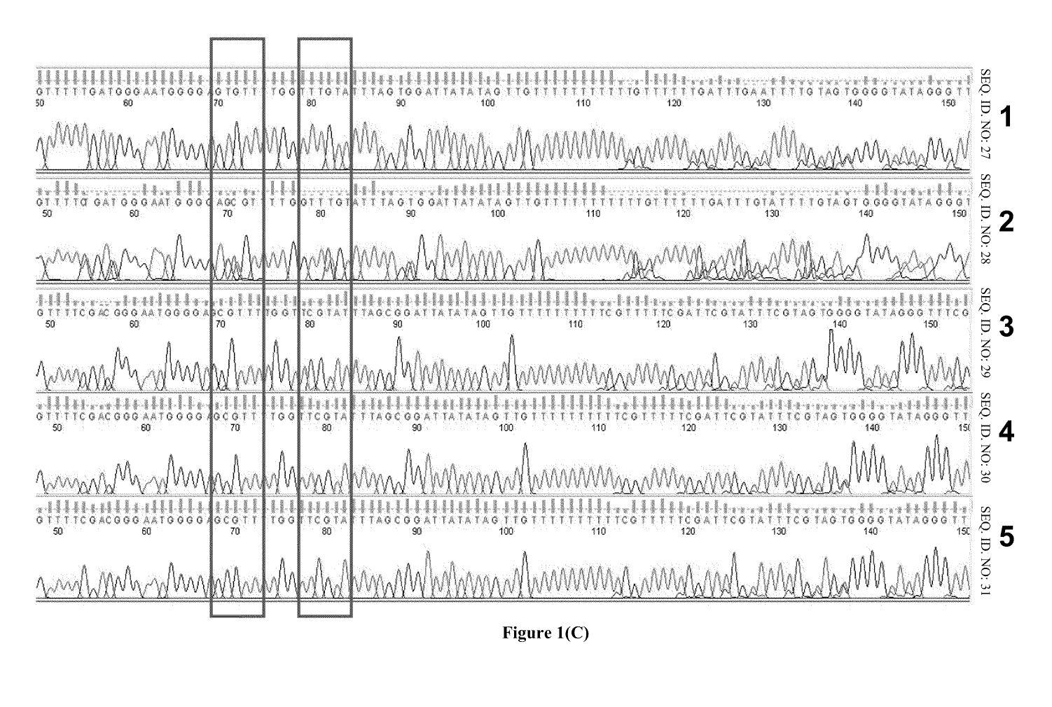

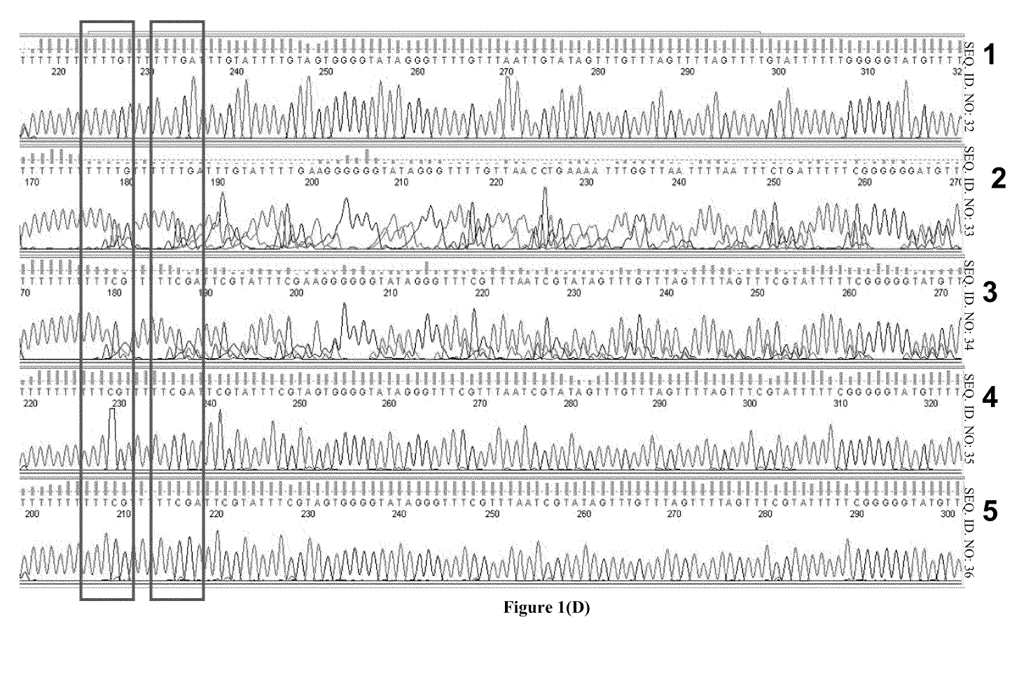

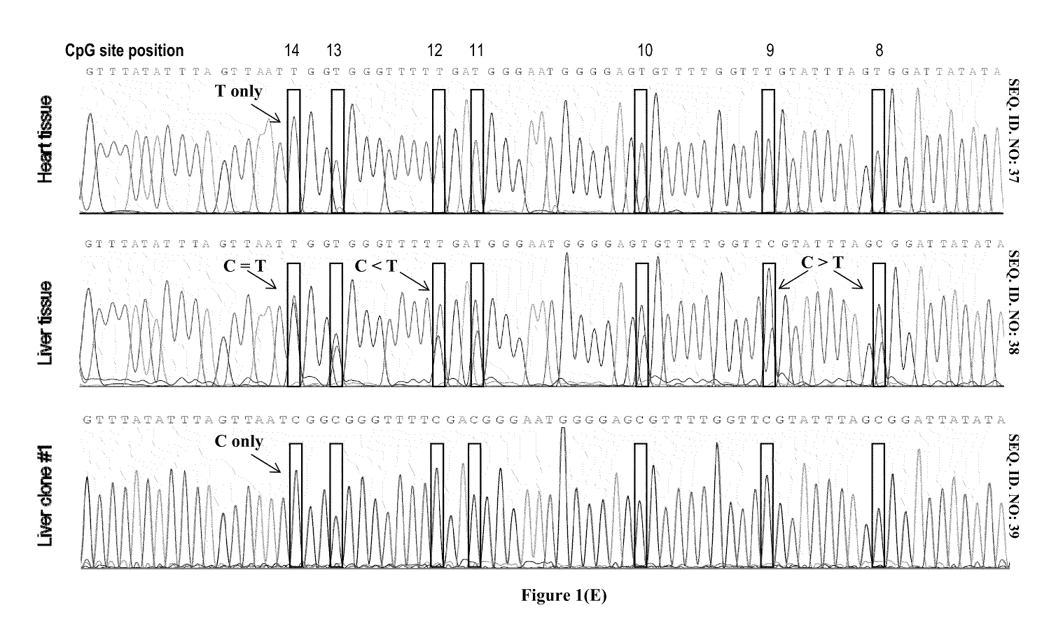

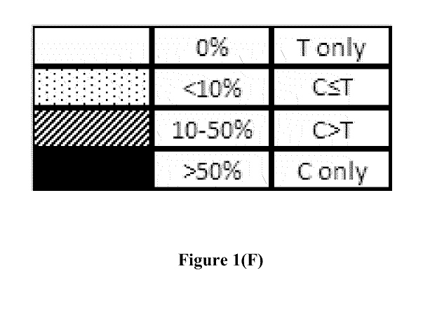

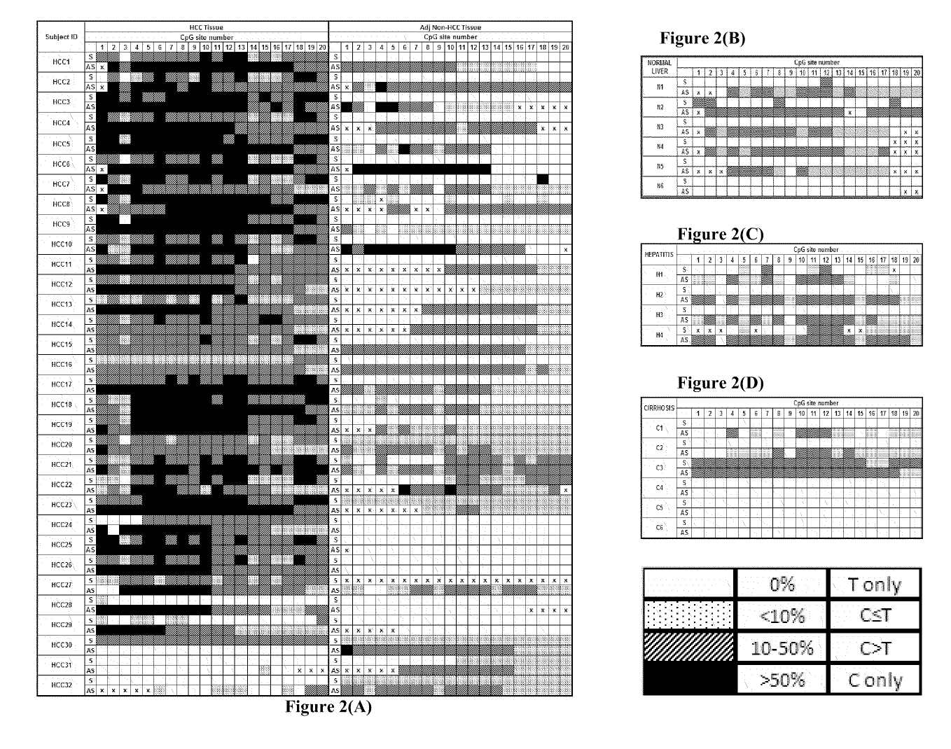

To determine the methylation profile of the sense and antisense strands of the APC gene, primers were designed for BSP and sequencing. FIG. 1A shows the CpG sites (vertical bars) on the promoter and first exon region of the APC gene and the primer locations for the bisulphite-specific primers. All 30 CpG sites within the 575-base pair region studied were numbered from 1 to 30 in the 5′ to 3′ direction. To control for the efficiency of the bisulphite conversion, the percentage of cytosine (C) to thymine (T) conversions were determined that occurred in non-CpG cytosines within the analyzed region after DNA sequencing of the BSP product from each sample. Only samples showing a C-to-T conversion rate higher than 95% for these non-CpG Cs were analyzed further. In order to compensate for the bias introduced in the sequencing results either by primer bias or software bias, the primers were first tested with spiked methylated DNA standards. (1) 100% unmethylated DNA, (2) 10% methylated DNA, 90% unmethylated DNA, (3) 25% methylated DNA, 75% unmethylated DNA, (4) 50% methylated DNA, 50% unmethylated DNA, (5) 100% methylated DNA. The chromatograms are shown in FIG. 1 (B, C, D). FIG. 1E shows the index used to determine the density of methylated CpG (^{m}CpG) sites. The ratio of the C peak to the T peak for each CpG site in the sequencing chromatogram derived from BSP sequencing, a semiquantitative methylation parameter, was used (23). The BSP sequencing chromatograms were divided into four categories: (1) C only (methylated, solid boxes); (2) C greater than T (hatched boxes); (3) C less than or equal to T (dotted boxes); and (4) T only (no methylation detected, open boxes). Based on FIG. 1 B-E, an index was created for semi-quantitative estimation of methylation (FIG. 1 F). Because the presence of one 16-base pair poly-T sequence in the antisense strand after bisulfite conversion rendered it unreadable by sequencing after CpG site 20, only CpG sites 1 to 20 were analyzed (FIG. 2).

BSP was carried out on bisulphite-treated DNA samples from HCC, matched adjacent non-HCC, normal liver, hepatitis-infected, and cirrhotic tissues followed by direct DNA sequencing of the PCR product. As expected, ^{m}CpG was detected in both the sense and antisense strands of the APC gene in most HCC DNA samples (27/32). Interestingly, an antisense-specific hemimethylation pattern in matched adjacent non-HCC tissue (19/32), in normal liver (5/6) samples, and in samples of liver from patients with hepatitis (3/4) and cirrhosis (2/6) was observed. The sense strand for these non-HCC tissues was unmethylated whereas the antisense strand showed variable densities of methylation. One cirrhosis sample, C3, showed symmetrical methylation, whereas two others, C4 and C5, showed no methylation (FIG. 2).

### Example 3

### Methylation Specific PCR Demonstrates Distinct Methylation Patterns in the Sense and Anti-Sense Strands of the APC Gene Promoter Region in Liver

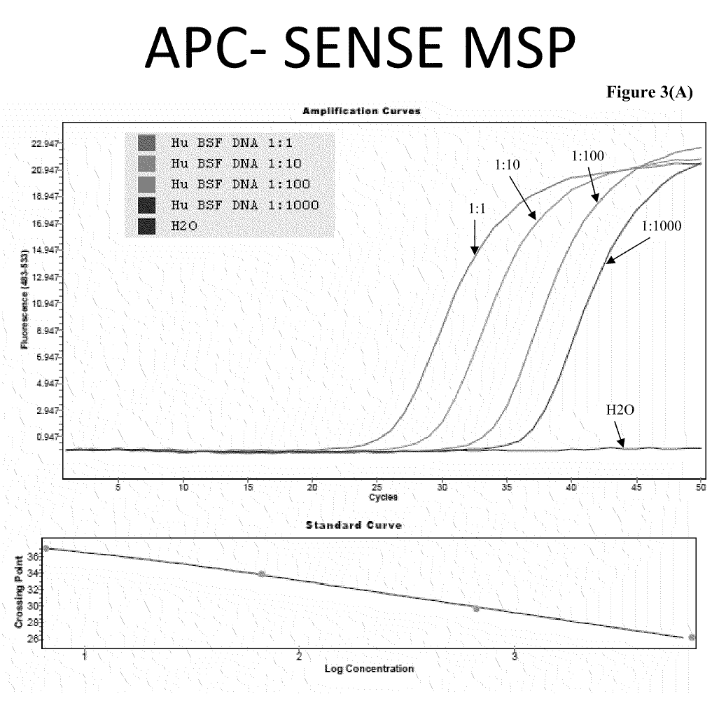

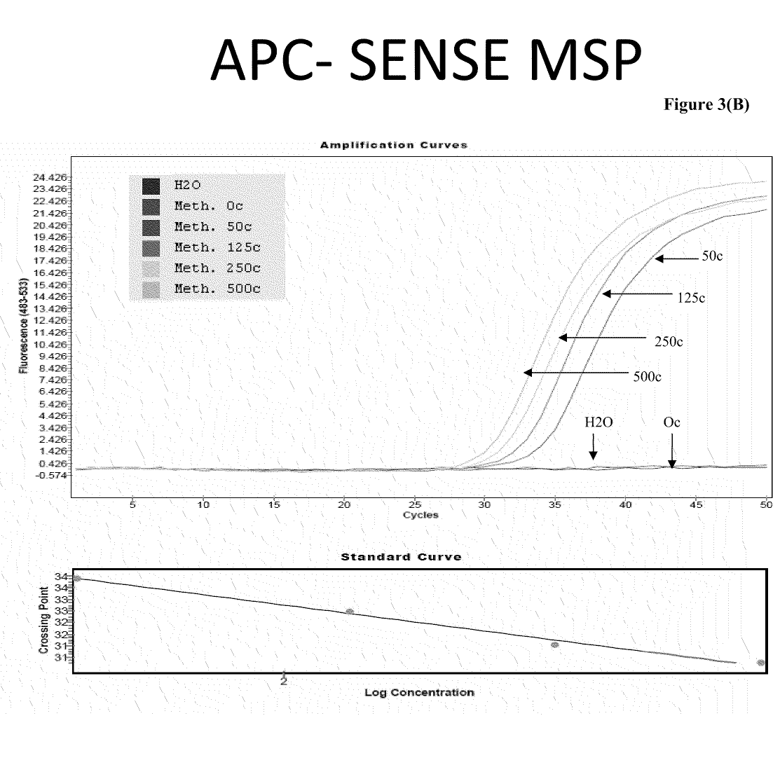

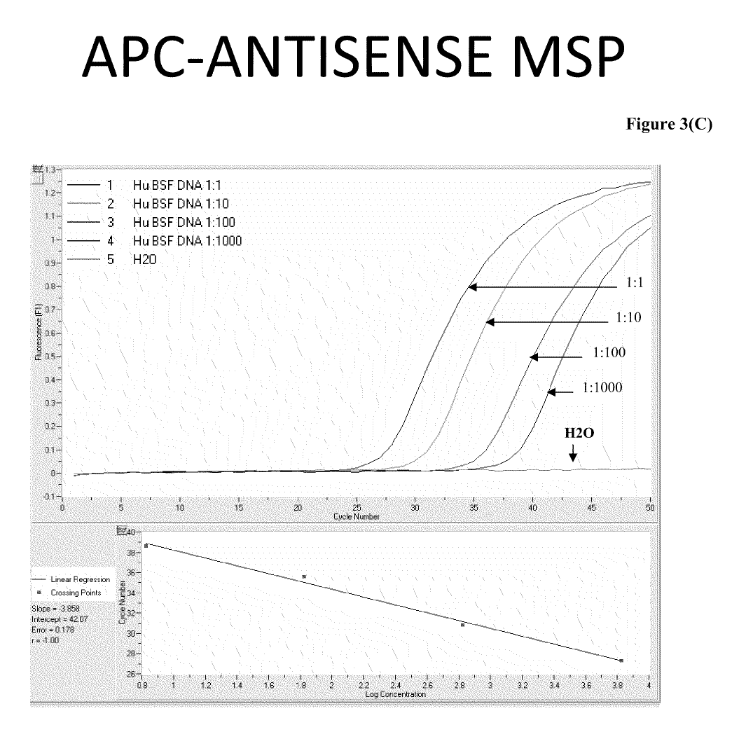

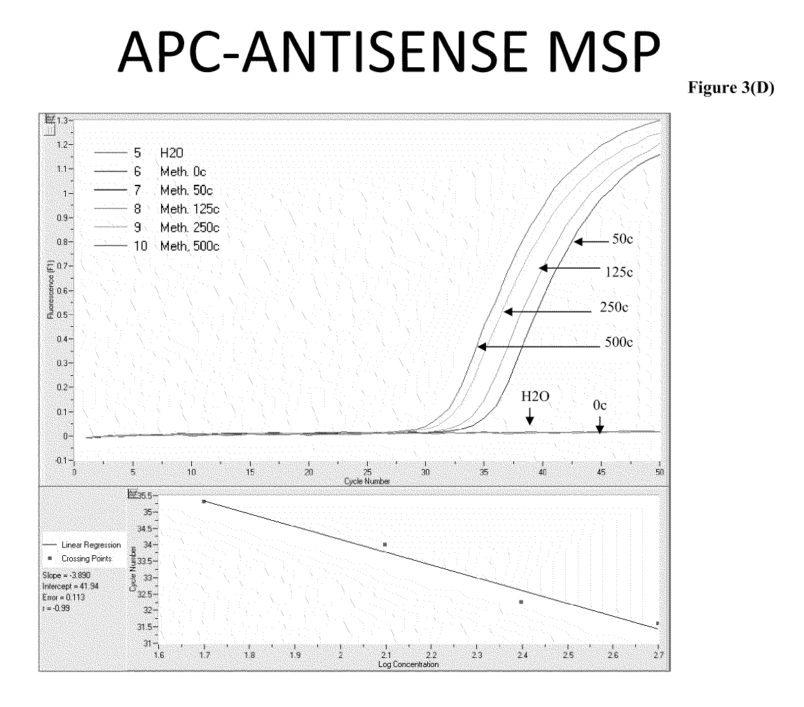

Two MSP assays were designed; one targeting CpG sites 7-17 on the sense strand and the other targeting CpG sites 11-18 on the anti-sense strand. The details regarding primer and Taqman probe sequences are in Table 4, above. These assays were tested against serial dilutions of human methylated bisulfite treated standards and spiked methylated DNA standards. Both the assays exhibit linear amplification characteristics with both sets of standards (FIG. 3). Bisulfite treated DNA from normal liver samples was tested (n=6), hepatitis (n=39), cirrhosis (n=41), adjacent non-HCC (n=58), and HCC samples (n=58). The data obtained was classified into 2 categories for each assay; methylation positive and methylation negative. The sense strand APC MSP showed higher specificity for HCC (92.31%) as compared to the anti-sense strand APC MSP (57.34%) (Table 5). The anti-sense strand MSP picked up methylated samples more frequently in non-HCC liver tissues and thus was less specific for HCC diagnosis.

TABLE 5
Sensitivity and specificity analysis by APC
sense strand and anti-sense strand MSP.

Methylation positive/Total

| Samples | Sense | Anti-sense |
|---|---|---|
| Normal | 1/5 | 0/5 |
| Hepatitis | 0/39 | 8/39 |
| Cirrhosis | 3/41 | 23/41 |
| Adjacent non-HCC | 7/58 | 30/58 |
| Total non-HCC | 11/143 | 61/143 |
| (normal, hepatitis, | | |
| cirrhosis, Adjacent | | |
| non-HCC) | | |
| HCC | 34/58 | 48/58 |
| Sensitivity % | 58.62% | 82.76% |
| Specificity % | 92.31% | 57.34% |

In all non-HCC liver tissues (normal, hepatitis, cirrhosis and adjacent non-HCC), the sense strand showed very low frequency of methylation while the anti-sense strand showed comparatively higher frequency of methylation. HCC liver tissues exhibited high level of methylation in both sense and anti-sense strands (FIG. 4).

Previous studies have shown an inconsistent association of methylation of the APC promoter region with HCC (Table 1, above). This comprehensive methylation study of the promoter and first exon region of the APC gene (FIGS. 2, 3, Table 5) suggests that all liver tissues, which include normal liver, hepatitis-infected, cirrhotic, and matched adjacent non-HCC and HCC, have methylation on their antisense strand. However, methylation observed on the sense strand appears to occur exclusively in HCC liver tissue.

### Example 4

### Methylation Density of the Antisense Strand in HCC Tissues was Significantly Higher than that in Non-HCC Tissues

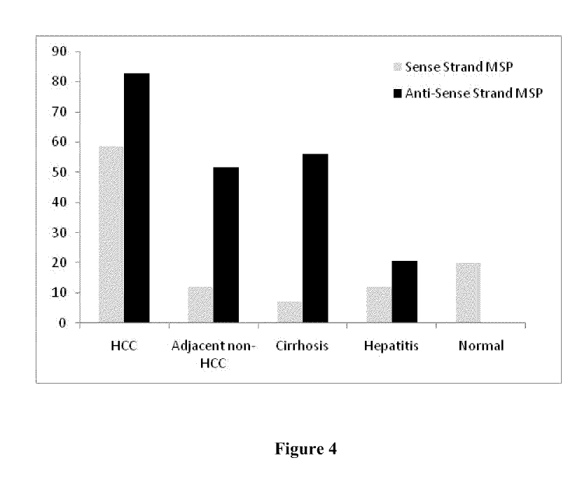

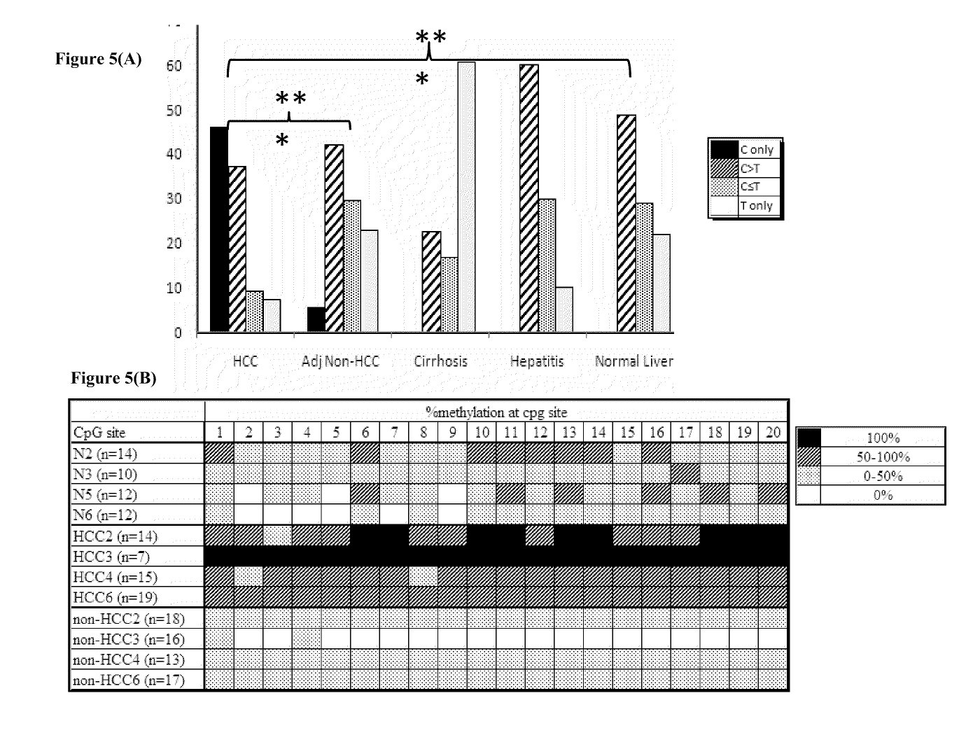

Next, the methylation density at the single CPG site level was analyzed of the antisense strand of the APC promoter and first exon region by BSP direct sequencing. No C only (filled boxes) was detected in normal or hepatitis-infected liver samples, whereas 46.1% (295/640) of CpG sites in HCC DNA and 5.4% (29/542) of CpG sites in matched non-HCC DNA showed C only in their antisense strand sequencing chromatograms (FIG. 5(A) and Table 6). The overall percentage of ^{m}CpG detected in the antisense strand of the APC gene among each disease category of liver tissue samples is summarized in Table 6. Methylation density of the antisense strand in HCC tissues was significantly higher than that in normal liver tissue or in adjacent non-HCC tissues (P<0.0001).

TABLE 6
Percent of ^{m}CpG detected in the antisense strand of the promoter and
the first exon region of the APC gene in each pathological group of liver tissue*

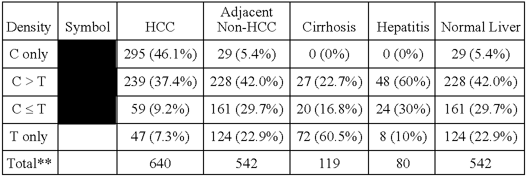

*The symbol and analysis of the methylation status of the CpG sites are described in FIG. 1(F). The percent of ^{m}CpG detected is calculated from the number of each category per pathological group. For example, 295 CpG sites were detected as “C only, filled box” among the total 640 sites analyzed in the HCC group (FIG. 2(A)); thus, the percent of “C only” is 46.2% in the HCC group.
**Total number of CpG sites analyzed.
APC, adenomatous polyposis coli; HCC, hepatocellular carcinoma; ^{m}CpG, methylated cytosine in a CpG dinucleotide

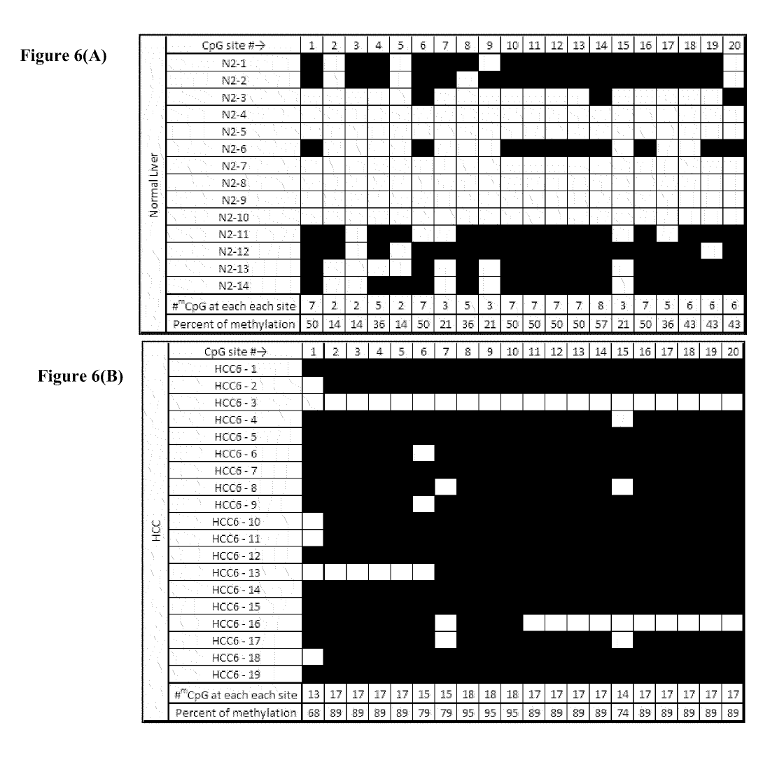

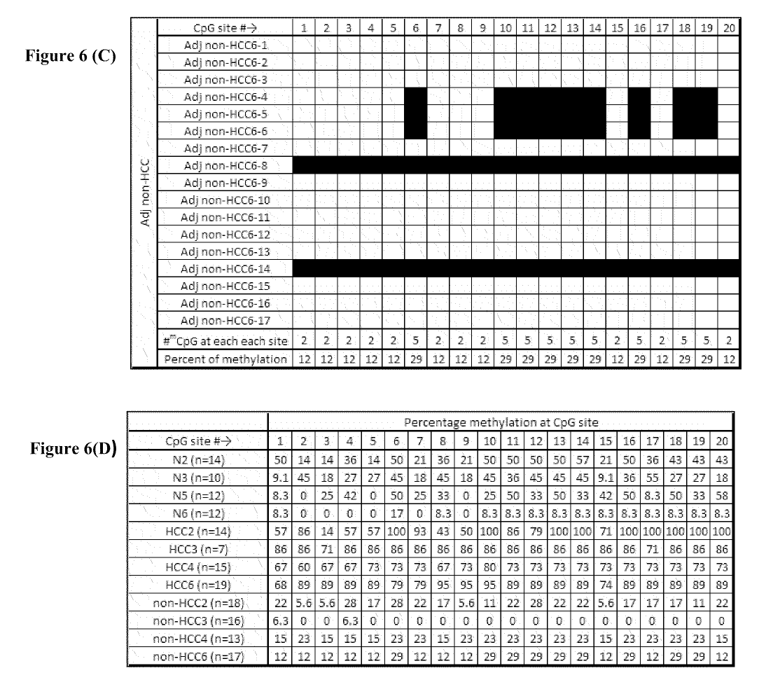

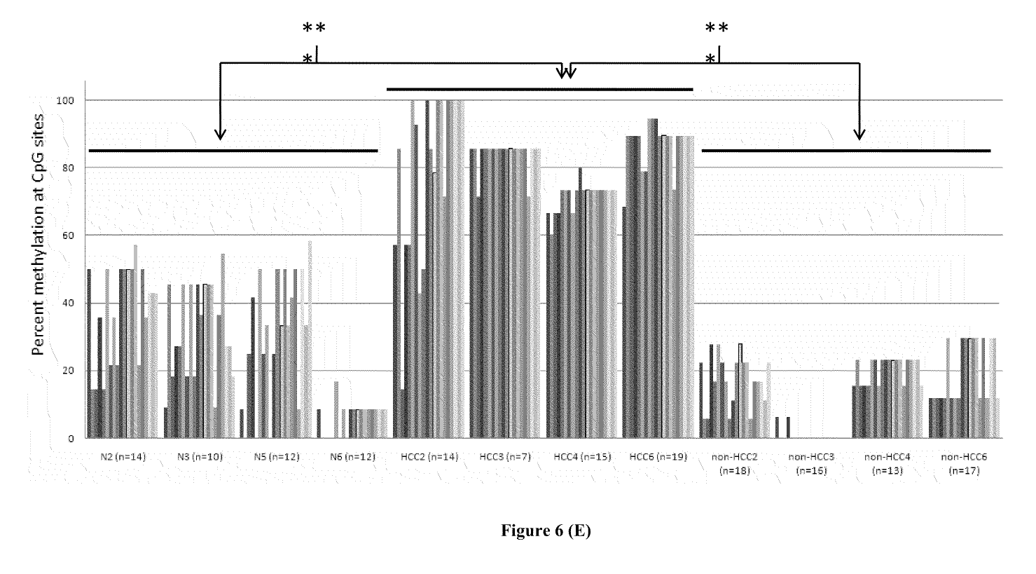

To further confirm that the density of ^{m}CpG in the antisense strand of APC increases in HCC, BSP cloning was performed and sequencing for the antisense strands from 4 normal livers, 4 HCC samples, and 4 matched adjacent non-HCC tissues. The percentage of clones that were methylated at each CpG site (percent methylation) in each DNA sample and in each tissue group was calculated (FIG. 5(B) and FIG. 6A-E). Data from BSP cloning and sequencing confirmed results from direct BSP sequencing: Methylation density of the antisense strand in HCC tissues was significantly higher than that in normal liver tissue and adjacent non-HCC tissue (P<0.0001) (FIG. 6(E)).

### Example 5

### Hairpin Bisulfite PCR Analysis of APC Promoter Region in Non-HCC Liver Tissue

The data shown so far is indicative of an anti-sense strand biased methylation pattern in non-HCC liver tissues. The next step was to confirm this by the hairpin bisulfite PCR that can provide the methylation status of the complementary strands from a single molecule.

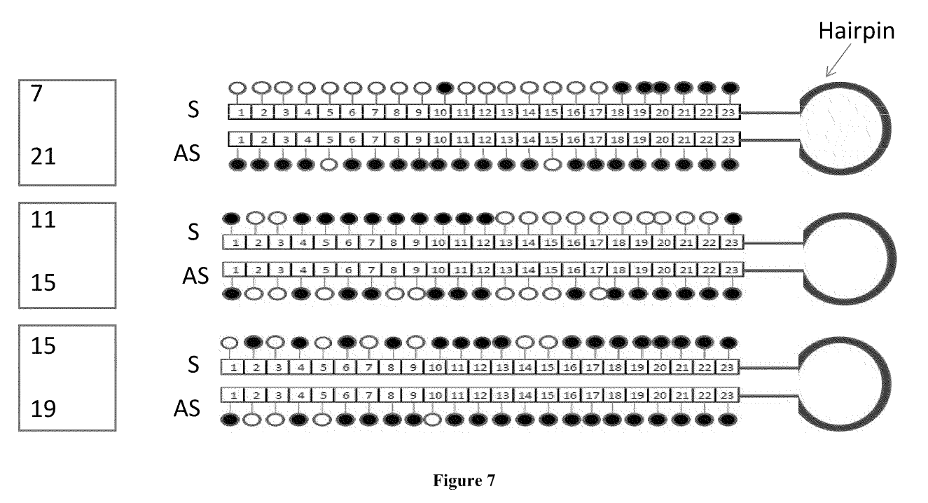

Two normal livers and adjacent non-HCC liver tissues were analyzed by hairpin bisulfite PCR, cloning the PCR product and sequencing. The Stratagene™ kit used previously with XL-Gold™ ultracompetent cells were unable to grow any colonies. A TOPO™ cloning kit with Top10™ chemically competent cells were used to clone this hairpin bisulfite PCR product. It was also observed that the clones obtained could grow only on solid agar and were unable to grow in liquid LB-broth. This suggested possible insert toxicity. Three hemimethylated clones were obtained after screening 17 total clones of adjacent non-HCC tissue (FIG. 7). The rest 13 clones were symmetrically methylated (data not shown). However, clones from the other 3 samples [1 adjacent no-HCC (n=17) and 2 normal livers (n=11 and n=16)] were all symmetrically methylated (data not shown).

### Example 6

### APC Hemimethylation is Liver Tissue Specific

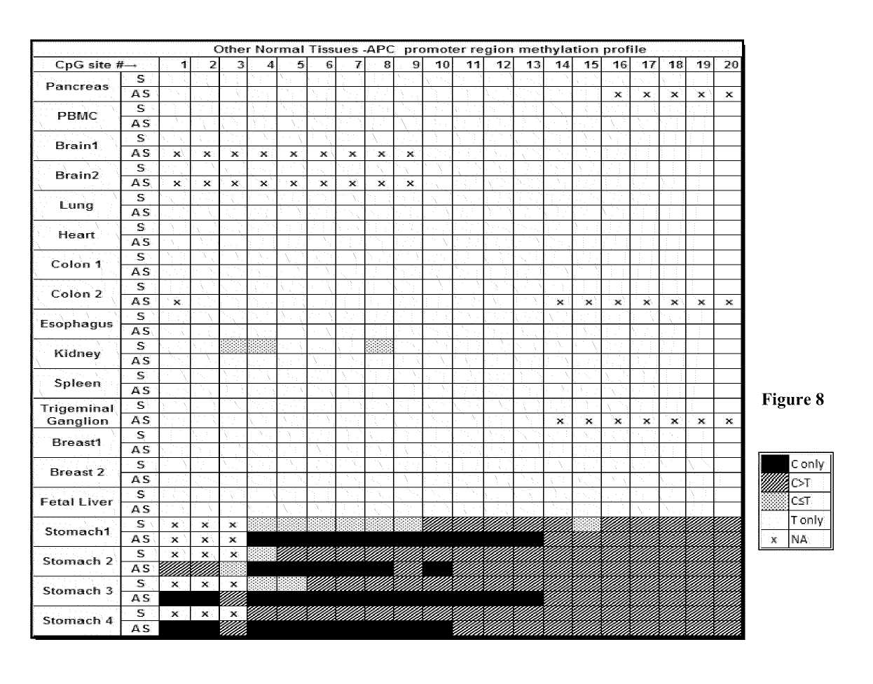

Because direct BSP sequencing indicates the net overall methylation status of DNA and avoids differential biases that can occur during bacterial cloning, BSP direct sequencing was applied to compare and analyze other tissue samples. To determine whether the observed findings were organ-specific, DNA isolated from thirteen different normal tissue types was analyzed: pancreas, peripheral blood mononuclear cells, brain, trigeminal ganglion, lung, heart, colon, esophagus, stomach, kidney, breast, spleen, and fetal liver (FIG. 8). Hemimethylation in any of the nonliver tissues or fetal liver tissue was not detected. Methylation of the promoter 1A of the APC gene occurred in normal gastric DNA in a monoallelic and age-dependent, but not antisense strand-specific, manner (24-27). By studying stomach tissue DNA from four individuals of different ages, 27, 29, 50, and 58 years, it was confirmed that the APC gene is methylated in normal gastric tissue DNA but in a symmetrical manner.

### Example 7

### DNA Methylation Patterns of GSTP1 and Actin in Liver

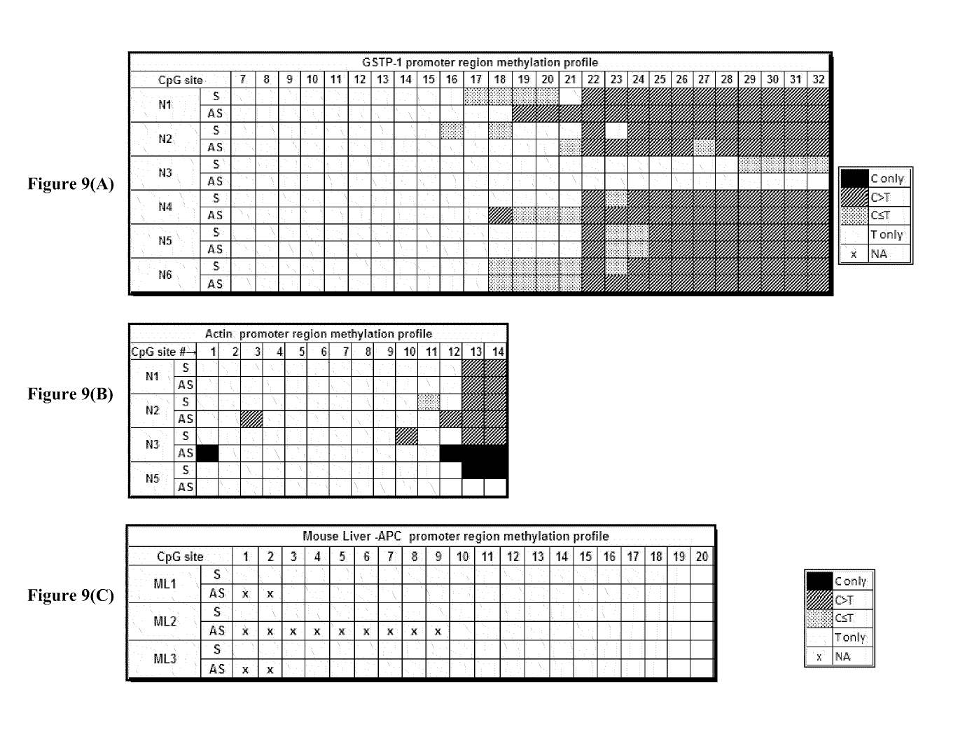

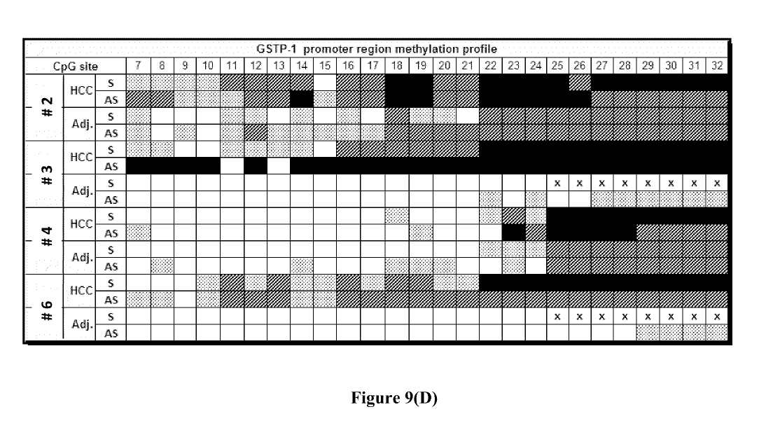

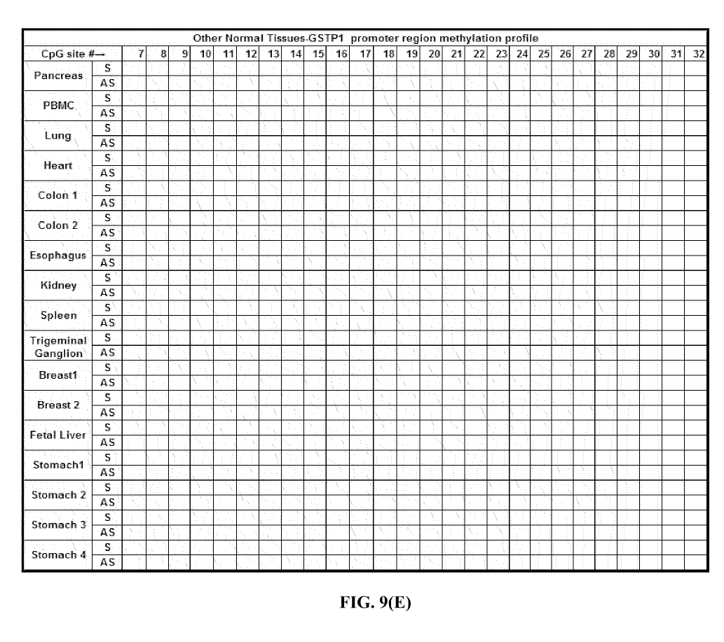

Because an antisense strand-specific CpG island hemimethylation pattern has not yet been reported in any human gene, it was sought to determine whether the observed event was gene-specific. Another known HCC-associated tumor suppressor gene, GSTP-1, as well as actin, a housekeeping gene, in liver tissues were investigated. Results revealed ^{m}CpG of both strands in the proximal GSTP-1 promoter and first exon in normal liver tissue (FIG. 9(A)). In the regions studied, symmetrical methylation of CpG sites occurred in 5 of 6 normal liver samples only in the part downstream of the −48 position from the start of the first exon. As expected, infrequent ^{m}CpGs were found in the promoter of the actin gene (FIG. 9(B)). These findings suggest that antisense-specific hemimethylation of APC is not generalized to all the genes in liver DNA. Mice are frequently used as animal models for studying human diseases, including cancers. As discussed, aberrant DNA methylation contributes to many cancers. Moreover, demethylating agents have been tested in mice as potential anticancer drugs (28). Thus, it was of interest to determine whether hemimethylation of the APC gene also occurs in mouse liver. Liver tissue DNA from three individual BALB/c female mice was subjected to BSP direct sequencing. No ^{m}CpG was detected in the three mice livers studied (FIG. 9(C)).

The partial symmetrical methylation of the GSTP1 promoter region was a surprising finding because a few published studies have suggested that methylation of the GSTP1 promoter region is an early detection marker for HCC (5, 6, 19, 21, 29). The GSTP1 methylation pattern in the five matched HCC and adjacent non-HCC tissues and in our panel of nonliver normal human tissues was examined. It was found that in three of five HCC and matched adjacent non-HCC tissues, methylation of CpGs occurred both upstream and downstream from the start of the first exon (FIG. 9D). Interestingly, no methylation was detected in the panel of other nonliver normal human tissues in the GSTP1 promoter region studied (FIG. 9E). Methylation of part of the promoter region in normal liver tissue but not in other nonliver tissue samples (FIGS. 9A, 9E) suggested another liver-restricted epigenetic pattern.

The entire disclosure of each patent, patent application, and publication cited or described in this document is hereby incorporated herein by reference.

### REFERENCES CITED

1. Ries L A G, Melbert D, Krapcho M, et al. (eds). SEER Cancer Statistics Review, 1975-2004, National Cancer Institute. Bethesda, Md., http://seer.cancer.gov/csr/1975_{—}2004/, based on November 2006 SEER data submission, posted to the SEER web site, 2007.
2. American Cancer Society. Cancer Facts & Figures 2010. Atlanta: American Cancer Society; 2010.
3. Jain S, Singhal S, Lee P, Xu R. Molecular genetics of hepatocellular neoplasia Am J Transl Res 2010; 2:105-118.
4. Lee S, Lee H J, Kim J H, Lee H S, Jang J J, Kang G H. Aberrant CpG island hypermethylation along multistep hepatocarcinogenesis. Am J Pathol 2003; 163:1371-1378.
5. Gao W, Kondo Y, Shen L, et al. Variable DNA methylation patterns associated with progression of disease in hepatocellular carcinomas. Carcinogenesis 2008; 29:1901-1910.
6. Moribe T, Iizuka N, Miura T, et al. Methylation of multiple genes as molecular markers for diagnosis of a small, well-differentiated hepatocellular carcinoma. Int J Cancer 2009; 125:388-397.
7. Nishida N, Nagasaka T, Nishimura T, Ikai I, Boland C R, Goel A. Aberrant methylation of multiple tumor suppressor genes in aging liver, chronic hepatitis, and hepatocellular carcinoma. Hepatology 2008; 47:908-918.
8. Lou C, Yang B, Gao Y, Wang Y, Fang S. Aberrant DNA methylation profile of hepatocellular carcinoma and surgically resected margin. Cancer Sci 2009; 100:996-1004.
9. Katoh H, Shibata T, Kokubu A, et al. Genetic inactivation of the APC gene contributes to the malignant progression of sporadic hepatocellular carcinoma: A case report. Genes Chromosomes Cancer 2006; 45:1050-1057.
10. Van der Auwera I, Bovie C, Svensson C, et al. Quantitative methylation profiling in tumor and matched morphologically normal tissues from breast cancer patients. BMC Cancer 2010; 10:97.
11. Virmani A K, Rathi A, Sathyanarayana U G, et al. Aberrant methylation of the adenomatous polyposis coli (APC) gene promoter 1A in breast and lung carcinomas. Clin Cancer Res 2001; 7:1998-2004.
12. Cedar H, Bergman Y. Linking DNA methylation and histone modification: patterns and paradigms. Nat Rev Genet 2009; 10:295-304.
13. Jones P A, Liang G. Rethinking how DNA methylation patterns are maintained. Nat Rev Genet 2009; 10:805-811.
14. Shi B, Vinyals A, Alia P, et al. Differential expression of MHC class II molecules in highly metastatic breast cancer cells is mediated by the regulation of the CIITA transcription: Implication of CIITA in tumor and metastasis development. Int J Biochem Cell Biol 2006; 38:544-562.
15. Burden A F, Manley N C, Clark A D, Gartler S M, Laird C D, Hansen R S. Hemimethylation and non-CpG methylation levels in a promoter region of human LINE-1 (L1) repeated elements. J Biol Chem 2005; 280:14413-14419.
16. Laird C D, Pleasant N D, Clark A D, et al. Hairpin-bisulfite PCR: Assessing epigenetic methylation patterns on complementary strands of individual DNA molecules. Proc Nat Acad Sci USA 2004; 101:204-209.
17. Singal R, vanWert J M. De novo methylation of an embryonic globin gene during normal development is strand specific and spreads from the proximal transcribed region. Blood 2001; 98:3441-3446.
18. Shao C, Lacey M, Dubeau L, Ehrlich M. Hemimethylation footprints of DNA demethylation in cancer. Epigenetics 2009; 4:165-175.
19. Woodcock D M, Lawler C B, Linsenmeyer M E, Doherty J P, Warren W D. Asymmetric methylation in the hypermethylated CpG promoter region of the human L1 retrotransposon. J Biol Chem 1997; 272:7810-7816.
20. Harder J, Opitz O G, Brabender J, et al. Quantitative promoter methylation analysis of hepatocellular carcinoma, cirrhotic and normal liver. Int J Cancer 2008; 122:2800-2804.
21. Csepregi A, Rocken C, Hoffmann J, et al. APC promoter methylation and protein expression in hepatocellular carcinoma. J Cancer Res Clin Oncol 2008; 134:579-589.
22. Feng Q, Stern J E, Hawes S E, Lu H, Jiang M, Kiviat N B. DNA methylation changes in normal liver tissues and hepatocellular carcinoma with different viral infection. Exp Mol Pathol 2010; 88:287-292.
23. Yang B, Guo M, Herman J G, Clark D P. Aberrant promoter methylation profiles of tumor suppressor genes in hepatocellular carcinoma. Am J Pathol 2003; 163:1101-1107.
24. Clark S J, Statham A, Stirzaker C, Molloy P L, Frommer M. DNA methylation: bisulphite modification and analysis. Nat Protoc 2006; 1:2353-2364.
25. Guilleret I, Osterheld M, Braunschweig R, Gastineau V, Taillens S, Benhattar J. Imprinting of tumor-suppressor genes in human placenta. Epigenetics 2009; 4:62-68.
26. Tsuchiya T, Tamura G, Sato K, et al. Distinct methylation patterns of two APC gene promoters in normal and cancerous gastric epithelia. Oncogene 2000; 19:3642-3646
27. Clement G, Bosman F T, Fontolliet C, Benhattar J. Monoallelic methylation of the APC promoter is altered in normal gastric mucosa associated with neoplastic lesions. Cancer Res 2004; 64:6867-6873.
28. Chan A O O, Lam S K, Wong B C-Y, Kwong Y-L, Rashid A, Tamura G. Gene methylation in non-neoplastic mucosa of gastric cancer: age or Helicobacter pylori related? Am J Pathol 2003; 163:370-373.
29. Qu Z, Fu J, Yan P, Hu J, Cheng S-Y, Xiao G. Epigenetic repression of PDZ-LIM domain-containing protein 2. J Biol Chem 2010; 285:11786-11792.
30. Chang H, Yi B, Li L, et al. Methylation of tumor associated genes in tissue and plasma samples from liver disease patients. Exp Mol Pathol 2008; 85:96-100.
31. Ferguson-Smith A C, Surani M A. Imprinting and the epigenetic asymmetry between parental genomes. Science 2001; 293:1086-1089.
32. Migeon B R. X-chromosome inactivation: molecular mechanisms and genetic consequences. Trends Genet 1994; 10:230-235.
33. Liang G, Chan M F, Tomigahara Y, et al. Cooperativity between DNA methyltransferases in the maintenance methylation of repetitive elements. Mol Cell Biol 2002; 22:480-491.
34. Robertson K D. DNA methylation and chromatin—unraveling the tangled web. Oncogene 2002; 21:5361-5379.
35. Issa J-P. Epigenetic variation and human disease. J Nutr 2002; 132:23885-2392.
36. Kwabi-Addo B, Chung W, Shen L, et al. Age-related DNA methylation changes in normal human prostate tissues. Clin Cancer Res 2007; 13:3796-3802.
37. Jones P A, Baylin S B. The fundamental role of epigenetic events in cancer. Nat Rev Genet 2002; 3:415-428.
38. Teschendorff A E, Menon U, Gentry-Maharaj A, et al. Age-dependent DNA methylation of genes that are suppressed in stem cells is a hallmark of cancer. Genome Res 2010; 20:440-446.
39. Penterman J, Zilberman D, Huh J H, Ballinger T, Henikoff S, Fischer R L. DNA demethylation in the Arabidopsis genome. Proc Nat Acad Sci USA 2007; 104:6752-6757.
40. Penterman J, Uzawa R, Fischer R L. Genetic interactions between DNA demethylation and methylation in Arabidopsis. Plant Physiol 2007; 145:1549-1557.
41. Wu S C, Zhang Y. Active DNA demethylation: many roads lead to Rome. Nat Rev Mol Cell Biol 2010; 11:607-620.
42. Kondo Y. Genetic instability and abberrant DNA methylation in chonic hepotitis and cirrhosis—A comprehensive study of loss of heterozygosity and microsatellite instability at 39 loci and DNA hypermethylation on 8 CpG islands in microdissected specimens from patients with hepatocellular carcinoma. Hepatology 2000; 32:970-979.
43. Deobagkar D D, Liebler M, Graessmann M, Graessmann A. Hemimethylation of DNA prevents chromatin expression. Proc Nat Acad Sci USA 1990; 87:1691-1695.
44. Maier H, Colbert J, Fitzsimmons D, Clark D R, Hagman J. Activation of the early B-cell-specific mb-1 (Ig-) gene by Pax-5 is dependent on an unmethylated Ets binding site. MolCell Biol 2003; 23:1946-1960.

What is claimed:

1. A method for detecting the presence or absence of a cancer in an individual, the method comprising:
determining the level of methylation of the sense strand of a selected regulatory region of a tumor suppressor gene from said individual;
comparing said level of methylation with the level of methylation found in one or more control samples from individuals known not to have said cancer; and
correlating a finding of elevated methylation in said individual as compared to the level of methylation in said one or more controls with an enhanced likelihood that said individual has said cancer.

2. The method of claim 1 wherein the cancer is hepatocellular carcinoma (HCC).

3. The method of claim 2 wherein the control is matched adjacent non-HCC sample.

4. The method of claim 1 wherein the tumor suppressor gene is adenomatous polypsis coli (APC).

5. The method of claim 1 wherein the regulatory region is the promoter of the APC gene.

6. The method of claim 1 wherein the regulatory region is the first exon of the APC gene.

7. The method of claim 1 wherein the regulatory region is the promoter and first exon of the APC gene.

8. The method of claim 1 wherein the individual is a human.

9. The method of claim 1 wherein determining the level of methylation of the sense strand is determining by methylation specific PCR (MSP).

10. The method of claim 9 wherein the methylation specific PCR (MSP) uses primers of the nucleotide sequences as set forth in SEQ ID NO: 15 and SEQ ID NO:16 and is probed by a probe of the nucleotide sequence as set forth in SEQ ID NO:17.

11. The method of claim 1 wherein determining the level of methylation of the sense strand is determining by bisulfite specific PCR (BSP) and sequencing.

12. The method of claim 11 wherein the bisulfite specific PCR (BSP) uses primers of the nucleotide sequences as set forth in SEQ ID NO: 1 and SEQ ID NO:2.

13. A method for detecting the presence or absence of a cancer in an individual, the method comprising:
determining if there is an apparent 100% methylation by assay of CpG sites in the anti-sense strand of a selected regulatory region of a tumor suppressor gene from said individual; and
correlating a finding of an apparent 100% methylation by assay in said individual with an enhanced likelihood that said individual has said cancer.

14. The method of claim 13 wherein the cancer is hepatocellular carcinoma (HCC).

15. The method of claim 13 wherein the tumor suppressor gene is adenomatous polypsis coli (APC).

16. The method of claim 13 wherein the regulatory region is the promoter and first exon of the APC gene.

17. The method of claim 13 wherein the individual is a human.

18. The method of claim 13 wherein determining the level of methylation of the sense strand is determining by bisulfite specific PCR (BSP) and sequencing.

19. The method of claim 18 wherein the bisulfite specific PCR (BSP) and sequencing uses primers of the nucleotide sequences as set forth in SEQ ID NO: 3 and SEQ ID NO: 4.

20. The method of claim 18 wherein the bisulfite specific PCR (BSP) and sequencing uses primers of the nucleotide sequences as set forth in SEQ ID NO: 5 and SEQ ID NO: 6.

21. A method for tissue typing, the method comprising:
determining the level of methylation of the anti-sense strand of a selected regulatory region of a tumor suppressor gene from said individual;
comparing said level of methylation with the level of methylation found in one or more control samples; and
correlating a finding of elevated methylation as compared to the level of methylation in said one or more controls with an enhanced likelihood that a tissue is liver.

22. The method of claim 21 wherein the tumor suppressor gene is adenomatous polypsis coli (APC).
